(19) United States
(12) Patent Application Publication
(10) Pub. No.: US 2009/0153801 A1
(43) Pub. Date: Jun. 18, 2009

(54) Light Projection Target Mire For Curvature Measurements

(76) Inventors: Liliane Ventura, Sao Paulo (BR); Cassius Riul, Sao Paulo (BR)

Correspondence Address: JOHN ALEXANDER GALBREATH, 2516 CHESTNUT WOODS CT, REISTERSTOWN, MD, 21136, US

(21) Appl. No.: 11/568,078

(22) PCT Filed: Apr. 15, 2005
(86) PCT No.: PCT/BR2005/000059

§ 371 Date: Oct. 19, 2006

(30) Foreign Application Priority Data
Apr. 22, 2004 (BR) PI0401628-9

(51) Int. Cl. A61B 3/10

(52) U.S. Cl. 351/212

(57) ABSTRACT

The present invention refers to the light ring target mire (8) used for accurate measuring of the radii of curvature of spherical and non spherical reflective surfaces, with a fixation target for the patient, holes that allow the observation by the slit lamp (7), holes to be illuminated (4), using an illumination system composed by a header which has an exit lens from the illumination system, focused filament, an intermediary circle and a light stop with an useful area for the target mire, being alternatively projected with arc shaped rings, or passing through rings in a precise circumlinear shape, allowing to be used with different types of illumination systems, providing measurements of any reflective surface, not being limited to ocular exams.

This invention is a device, luminous ring target, specially developed and designed to be used for precision measurements of the radii of curvature of reflecting surfaces of any shape, having 72 holes for light projection. The target may alternatively have any number of holes. There are also two additional holes that allow the observation through the eyepieces of the slit lamp and any ocular microscope and any other device to which the target is coupled. The target may also be alternatively built with continuous ring for light projection, as well as arc shaped rings in a precise circumlinear shape. The target uses external illumination provided by the light bulb of the slit lamp and may be used for any kind of external illumination.

Similarly, if several additional holes (or arc shaped, or continuous ring shape) are to be made along the device, it allows measurements of the topography of any reflecting surface, specially the cornea.

Two conical surfaces, having accurately determined angles, have been designed for providing uniform reflection of the light, in order to reflect the greatest amount of light, providing the desired illuminated diameter of the target, considering the focusing of the light from the illumination system of the slit lamp and thus its angulation, which is relevant to reflect the light to the desired direction.

The invention has been built to work using the illumination system of the slit lamp and/or any microscope lamp or focused light source within a system of double reflection or the light in conic polished and/or mirrored surfaces, providing the homogeneous complete illumination of the target mire.

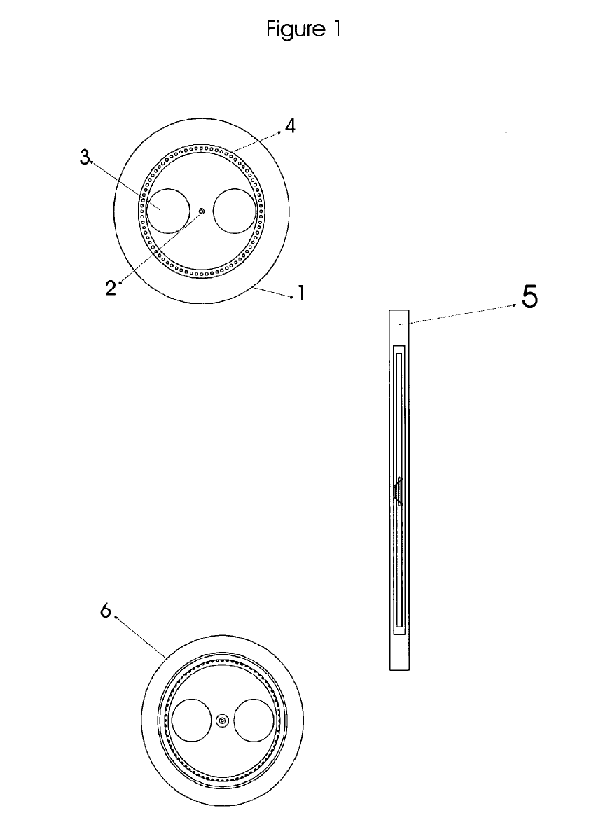

The aim of the invention is, therefore, to illuminate the pin-holes showed in FIG. 1—holes to be illuminated in a number of 06 or more—with the light from the slit lamp.

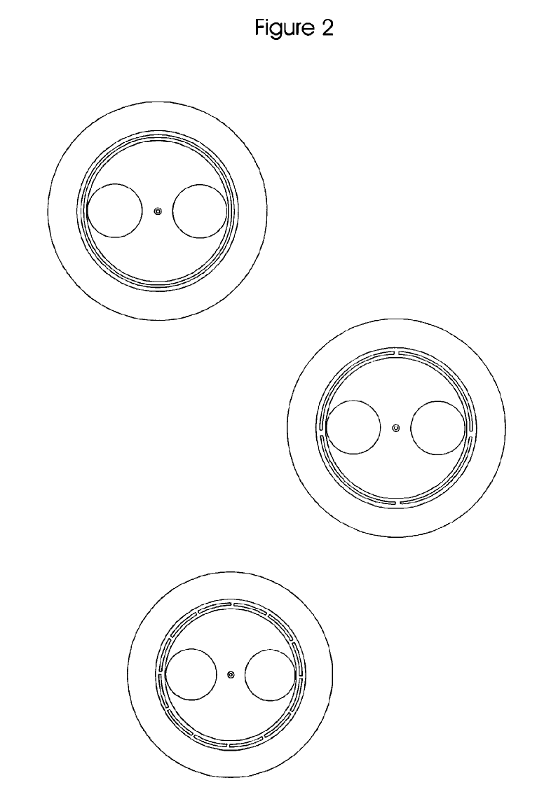

Alternatively to the pin-holes, arcs or a ring shape target may be used as targets in a accurate circumlinear shape, as shown in FIG. 2.

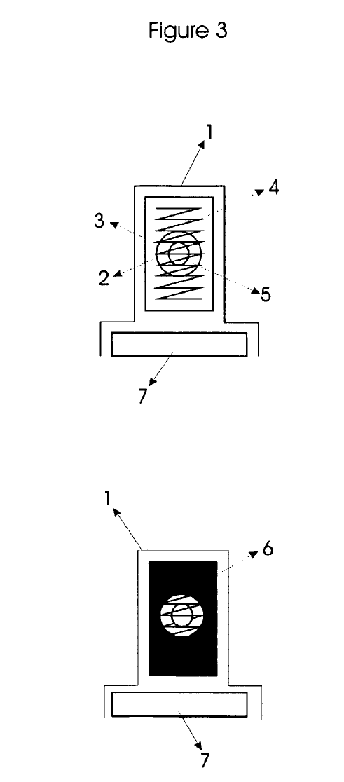

The light filament of the illumination system of the slit lamp is focused within the exit pupil from its own illumination device, refer to FIG. 3.

The calculation for manufacturing the mire target takes into account the selected region, as the most external circle, and it is called “useful area spot” (4), presented on FIG. 3a. A better illumination homogeneity may be obtained using a light stop as indicated on FIG. 3b.

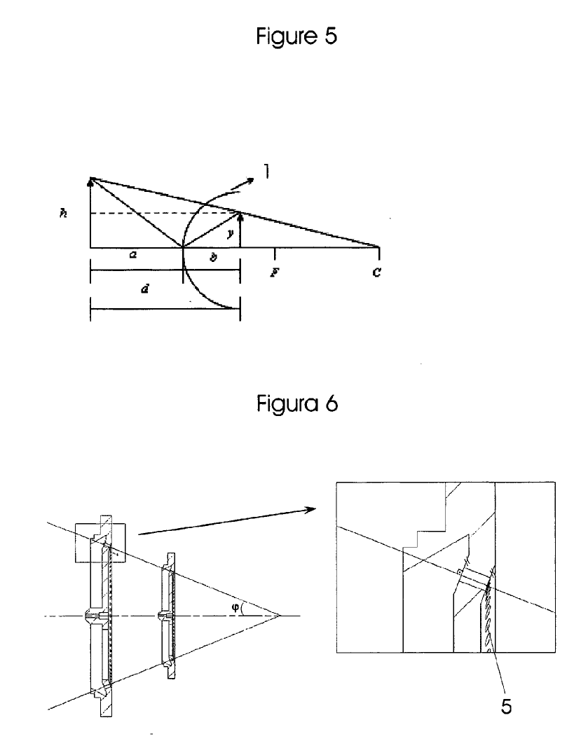

As the focal distance “F” (refer to FIG. 5) for the light rays that emanates from the system is known and as a spot of the projected filament may be selected, it is possible to draw an imaginary ray from this spot to the focal point.

For calculus optimization reasons, this spot will be taken from the “intermediary circle region” (5) shown on FIG. 3. Therefore, it becomes easy to obtain the focusing angle between this ray and the perpendicular axis to the illumination system that passes by the focus spot. It its then possible to deviate this light ray, by the reflecting surfaces, to an intended position.

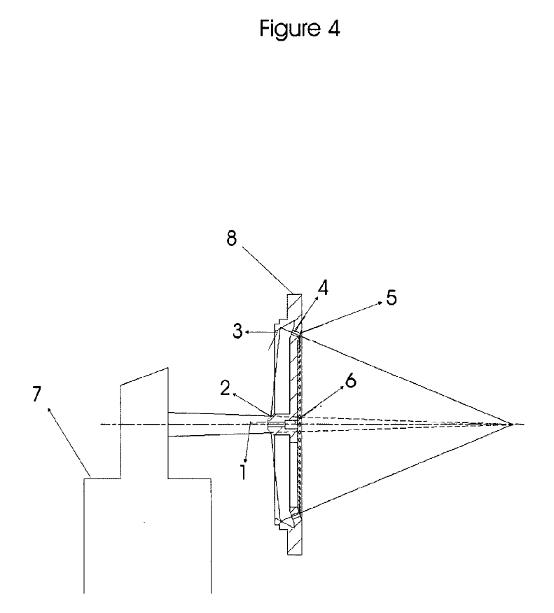

The face indicated by (1) on FIG. 4 is positioned on the central axis of focalization of the optical system. In the center of this face there is a hole for light passing, which will be a reference for the patient's fixation spot. Face 2 is a mirrored polished conic surface that reflects the light that comes from the illumination system toward to another reflecting surface 3.

Surface 3 is the internal surface of a conic section that deviates light for illuminating the pin-holes on face 4, that now become small light spots sources having their directed to the original focus of the light of the slit lamp.

The relevance of this issue is not the patient's cornea illumination, but the homogeneous illumination of the pin-holes (4) of the target mire, since what is relevant is the reflection of the illuminated target by the lachrymal surface of the cornea or any reflective surface under test. In this sense, the focusing of the light spots is necessary for the system's optimization, for providing an accurate focusing distance and consequently a most effective center positioning during the exam.

The target mire manufacturing details are presented in FIGS. 4, 6, 7 and 8.

FIG. 4 shows the target mire placed on the slit lamp. The continuous and dashed lines represent the light rays tracing that reach surface 2.

If the mire target did not intercept the light rays, the rays would reach the original focus position of the system, as shown by the dashed lines.

When the target mire intercepts the light path, the ray is deviated toward surface 3 and then again to toward the slit lamp's focus position. Therefore, the pin-holes in 4 are focused onto the patient's cornea.

The way that the holes are illuminated by the focused light rays that are at precise pre-determined angles for their reflection is essentially innovating by homogeneously illuminate the said holes and also to project at the reflective surface (cornea) the circular target mire, with homogeneous illumination and accurate dimension.

The reflective properties of the cornea are similar to the spherical mirror. Considering the cornea as a spherical mirror, the equation that defines the diameter of the target mire may be obtained as a function of the desired size of the projected image. For instance, in keratometry measurements, the size of the projected image of the mire onto the cornea is 3 mm for a non astigmatic standard eye.

From the geometric properties of FIG. 5, the diameter of the target mire may be written as:

$$\begin{matrix}h = \frac{2 yd \pm 2 y \sqrt{{d}^{2} - {R}^{2}}}{2 R} & ( 1 ) \\ \frac{h}{a} = \frac{y}{b} and \frac{h - y}{d} = \frac{y}{R - b} & ( 2 )\end{matrix}$$

where 2h is the diameter of the target mire and 2y is the diameter of the projected image onto the reflective surface (cornea).

The diameter of the invention is specific image size dependent and the distance between the target mire and the eye dependent. Observing an imaginary dashed cone, shown in FIG. 6, and having the image size that it is pre-determined by the designer, the diameter where the holes (6) of the conic surface should be made are determined.

In order to project the reflective surfaces of the target mire, the imaginary ray coming from the intermediary circle (5) in FIG. 3, should be considered.

The geometric calculus are done considering the rays coming from the intermediary circle, that reach the central parts of the reflective surfaces in order to reflect the light rays that come from the useful parts on surfaces 2 and 3 (reflection angle is the same as the incidence angle-reflection law) and to reach surface 4.

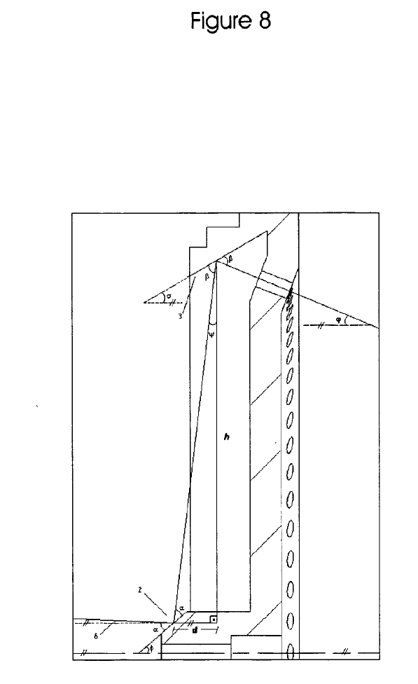

At surface 2, the reflective angle is α and at surface 3, the reflective angle is β. Once distance “d” and the image size 2h are defined between the incidence points 2 and 3, angles α, β and σ are obtained by the equations (1), (2), (3), (4), (5) and (6) (refer to FIG. 8):

$$\begin{matrix}α = \frac{90 + δ - arc tg \frac{d}{h}}{2} & ( 3 ) \\ β = \frac{270 - ϕ - arc tg \frac{d}{h}}{2} & ( 4 ) \\ φ = α - δ & ( 5 ) \\ σ = 90 - β - arc tg \frac{d}{h} & ( 6 )\end{matrix}$$

The set up of the invention is quite simple, with no level of difficulty for the user, mainly because of its cables free design, consisting in just easily adapting it at the illumination header of the slit lamp.

Regarding the state of the art of the invention, no similar device has been earlier published. Hence, the present invention has full filled all the requirements for the present patent that consists of a light target mire that should replace the previous developed target mires for keratometric measurements and/or determination of the radii of curvature of any reflective surface of patent number P103005483-7 in Jul. 7, 2003 and PCT/BR 03/00200, in Dec. 19, 2003, which have no similarity to the present invention, either in its project or design, which is totally innovatory.

The main advantages and features of the present invention is its significantly reduced size, better location and easy adaptation at the Slit Lamp, its independence from extra light sources, since it uses the light coming from the Slit Lamp or any other focused light source, reflecting the light and providing an image of a light ring similar to the previous said patent.

In order to provide a clear visualization of the present invention, the illustrative drawings are attached:

FIG. 1 represents the invention and its frontal view (1) with the fixation target for the patient (2), holes that allow the observation through the slit lamp (3), holes to be illuminated (4), side view and anterior view (6).

FIG. 2 presents a front view of an alternative target mire to be illuminated having arcs or passing through rigs in an accurate circular shape.

FIG. 3 presents the front view of the illumination header of the slit lamp (1) exit lens (2) of the illumination system (a), projected filament (3), useful light spot for the target mire (4), intermediary circle (5) light stop (6).

FIG. 4 presents the illumination system of the slit lamp (7) illuminating the projections target mire (8) having a face (5) placed perpendicularly to the focal point of the illumination system and a pin-hole as a fixation target for the patient (6), having the entrance pupil for the light manufactured on the front face (10) and the exit pupil having a larger diameter, that may be alterated depending on the desired size of the spot as a fixation target for the patient, a reflective conic surface (2), inner part of a conic surface (3) that illuminates the pin holes, arcs or passing through ring (4).

FIG. 5 shows the schematic drawing of the projection image of the target mire on the reflective surface for radii of curvature calculations purposes (1) and respective geometric angles.

FIG. 6 presents the drawing of two alternative target mires, having distinct diameter and projecting the same image size onto the reflective surface, since the target mire is in the inner part of the imaginary cone represented in FIG. 6 by dashed lines, highlighting the conic surface that contains the holes (5) (or passing through arcs or circular ring) represented by a rectangle.

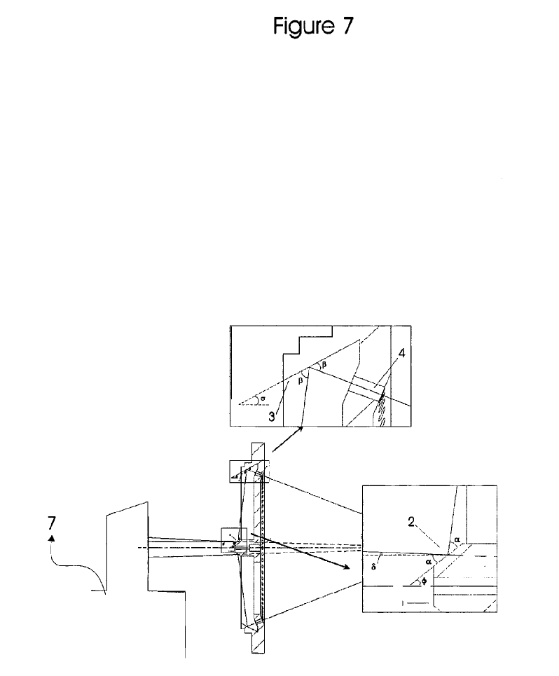

FIG. 7 presents the drawing of the reflective surface (4), which deviates the light in order to illuminate the pin holes (5), showing in details the conic reflective surface (2) as well as the illumination header (a) of the slit lamp (2).

FIG. 8 shows the light path in the target mire due to the light rays reflection.

The schematic drawings of the present invention are merely illustrative and changes may be performed in its details, specially in its size, shape and dimension to the extension provided by the knowledge of the present claims, but always into the innovative principle, having the invention to incorporate innovatory features, with commercial and manufacturing applications, where the present invention fulfills these requirements, having singular principles in comparison to the other products, due to its advantages features, technical effect impact and that have all the necessary conditions to reach the required privilege.

1) “LIGHT PROJECTION TARGET MIRE FOR ACCURATE MEASUREMENTS OF THE RADII OF CURVATURE OF SPHERICAL AND NON SPHERICAL REFLECTING SURFACES”, characterized by a device, light ring target, used for precise measurements of the radii of curvature or reflective surfaces of any shape, having 72 holes, more than or less than, for light projection and two additional holes that allow the observation through the eyepieces of the slit lamp or any other ocular microscope or any other device to which the target may be attached, externally illuminated by a light or any other type of illumination, in particular, for the slit lamp that has an illumination system composed by header which has a light bulb, an exit lens from the illumination system, focused filament, an intermediary circle and a light stop with an useful area for the target mire, being alternatively projected with arc shaped rings, or passing through rings in a precise circumlinear shape, allowing to be used with different types of illumination systems, providing measurements of any reflective surface, not being limited to ocular exams.

2) “LIGHT PROJECTION TARGET MIRE FOR ACCURATE MEASUREMENTS OF THE RADII OF CURVATURE OF SPHERICAL AND NON SPHERICAL REFLECTING SURFACES” according to claim 1, is characterized by the way that the holes are illuminated by the focused light rays that are at precise pre-determined angles for their reflection and homogeneously illuminate the said holes and also project at the reflective surface the circular target mire, with homogeneous illumination and accurate dimension.

3) “LIGHT PROJECTION TARGET MIRE FOR ACCURATE MEASUREMENTS OF THE RADII OF CURVATURE OF SPHERICAL AND NON SPHERICAL SURFACES” according to claim 1, is characterized by holes (or passing through arcs or rings) positioned circularly that allow to create a projection mire of many light ring targets composed by illuminated light spots (or continuous rings, or arcs) which allow to determine the curvature radii of the reflective surface, in this case, the cornea.

4) “LIGHT PROJECTION TARGET MIRE FOR ACCURATE MEASUREMENTS OF THE RADII OF CURVATURE OF SPHERICAL AND NON SPHERICAL REFLECTING SURFACES”, according to claim 1, is characterized by two conical surfaces, having accurately determined angles, designed for providing uniform reflection of the light, in order to reflect the greatest amount of light, providing the desired illuminated diameter of the target, considering the focusing of the light from the illumination system of the slit lamp and thus its angulation, which is relevant to reflect the light to the desired direction and may be designed to work within any other focused and projected light source, with the intention of deviating the light circularly for obtaining the homogeneous and total illumination of the target mire.

5) “LIGHT PROJECTION TARGET MIRE FOR ACCURATE MEASUREMENTS OF THE RADII OF CURVATURE OF SPHERICAL AND NON SPHERICAL REFLECTING SURFACES”, according to claims 1, 2, 3, 4 and 5, is characterized by the geometric calculus considering the rays coming from the intermediary circle, that reach the central parts of the reflective surfaces in order to reflect the light rays that come from the useful parts on surfaces 2 and 3 (reflection angle is the same as the incidence angle-reflection law) and to reach surface 4 at reflective angle α at surface 2, and at reflective angle β on surface 3, by the reflection law, will be reflected at the same incidence angle and defining distance “d” and the image size 2h between the incidence points 2 and 3, angles α, β and σ are obtained by the equations (1), (2), (3), (4), (5) and (6):
$$\begin{matrix}α = \frac{90 + δ - arc tg \frac{d}{h}}{2} & ( 3 ) \\ β = \frac{270 - ϕ - arc tg \frac{d}{h}}{2} & ( 4 ) \\ φ = α - δ & ( 5 ) \\ σ = 90 - β - arc tg \frac{d}{h} & ( 6 )\end{matrix}$$
